(19) United States
(12) Patent Application Publication
(10) Pub. No.: US 2022/0142661 A1
(43) Pub. Date: May 12, 2022

(54) Medical Systems and Methods Thereof for Ultrasonic Decomposition of Intraluminal Clots

(71) Applicant: Bard Access Systems, Inc., Salt Lake City, UT (US)

(72) Inventors: William Robert McLaughlin, Bountiful, UT (US); Steffan Sowards, Salt Lake City, UT (US); Devan Anderson, Taylorsville, UT (US); Anthony K. Misener, Bountiful, UT (US)

(21) Appl. No.: 17/524,455

(22) Filed: Nov. 11, 2021

Related U.S. Application Data
(60) Provisional application No. 63/113,074, filed on Nov. 12, 2020.

(51) Int. Cl. A61B 17/22

(52) U.S. Cl. CPC A61B 17/2202; A61B 2017/22028; A61B 2017/22021; A61B 2017/22007

(57) ABSTRACT

Disclosed herein are medical systems and methods for ultrasonic decomposition of intraluminal clots. A medical system can include a stylet configured to insert into a lumen of a catheter. The stylet can include one or more electrical impedance sensors, as well as one or more ultrasound transducers or a resonant section of the stylet. The one-or-more impedance sensors can be configured to detect changes in impedance for identifying intraluminal clots in the catheter. The one-or-more ultrasound transducers can be configured for decomposing the intraluminal clots to reestablish patency in the catheter, while the resonant section of the stylet can be configured to resonate with an externally applied ultrasonic frequency for decomposing the intraluminal clots to reestablish patency in the catheter. The medical system can further include a console to which the stylet is connected in an operable state of the medical system.

### PRIORITY

This application claims the benefit of priority to U.S. Provisional Patent Application No. 63/113,074, filed Nov. 12, 2020, which is incorporated by reference in its entirety into this application.

### BACKGROUND

Thromboses, otherwise known as blood clots, can occur in blood vessels in which catheters such as peripheral intravenous catheters (“PIVCs”), peripherally inserted central catheters (“PICCs”), central venous catheters (“CVCs”), or the like are commonly placed. Like thromboses in blood vessels, intraluminal clots can occur in lumens of such catheters. The intraluminal clots are commonly treated with thrombolytic drugs such as alteplase, which is tissue plasminogen activator (“TPA”) produced by recombinant DNA technology. TPA catalyzes conversion of an open form of clot-bound plasminogen into active plasmin, which is a major enzyme responsible for breaking down fibrin in thromboses.

In treating an intraluminal clot to restore patency to a catheter, a clinician typically forces a solution of TPA into a lumen of the catheter proximal of the intraluminal clot using a 3-way stopcock method. The method requires a drawing step of drawing fluid from the lumen of the catheter proximal of the clot with a first syringe, thereby creating a partial vacuum, an injecting step of injecting the solution of TPA into the lumen of the catheter under the vacuum with a second syringe, and a waiting step of waiting up to at least 20 minutes for the TPA to act on the intraluminal clot before repeating the foregoing steps. While the 3-way stopcock method can be effective, it can also take over an hour or more to treat an intraluminal clot due to its size, extent of occlusion, and location in the catheter.

Disclosed herein are medical systems and methods thereof for ultrasonic decomposition of intraluminal clots that improve upon at least the 3-way stopcock method.

### SUMMARY

Disclosed herein is a medical system for ultrasonic decomposition of intraluminal clots. The medical system includes, in some embodiments, a stylet. The stylet is configured to insert into a lumen of a catheter. The stylet includes one or more electrical impedance sensors and one or more ultrasound transducers. The one-or-more impedance sensors are in a distal portion of the stylet. The one-or more impedance sensors are configured to detect changes in impedance for identifying intraluminal clots in the catheter. The one-or-more ultrasound transducers are embedded in the distal portion of the stylet. The one-or-more ultrasound transducers are configured for decomposing the intraluminal clots to reestablish patency in the catheter.

In some embodiments, the one-or-more ultrasound transducers form an ultrasound-transducer array embedded along a length of stylet.

In some embodiments, the one-or-more ultrasound transducers form an ultrasound-transducer array embedded around a circumference of the stylet.

In some embodiments, the one-or-more ultrasound transducers are configured for decomposing the intraluminal clots by ultrasonic cavitation of fluid proximate of the intraluminal clots.

In some embodiments, the one-or-more ultrasound transducers are further configured for decomposing the intraluminal clots by ultrasonic agitation of a thrombolytic drug in the fluid proximate the intraluminal clots.

In some embodiments, the one-or-more ultrasound transducers are configured for decomposing the intraluminal clots by direct contact of the intraluminal clots with the stylet while the one-or-more ultrasound transducers are in operation.

In some embodiments, the one-or-more ultrasound transducers are configured to automatically activate upon identification of the intraluminal clots by the changes in impedance in accordance with logic onboard a console to which the stylet is connected in an operable state of the medical system.

Also disclosed herein is another medical system for ultrasonic decomposition of intraluminal clots. The medical system includes, in some embodiments, a stylet and an ultrasound probe. The stylet is configured to insert into a lumen of a catheter. The stylet includes one or more electrical impedance sensors and a resonant section of the stylet. The one-or-more impedance sensors are in a distal portion of the stylet. The one-or-more impedance-sensors are configured to detect changes in impedance for identifying intraluminal clots in the catheter. The resonant section of the stylet is in the distal portion of the stylet distal of the one-or-more impedance sensors. The resonant section of the stylet is configured to resonate with an externally applied ultrasonic frequency for decomposing the intraluminal clots to reestablish patency in the catheter. The ultrasound probe is configured to apply the ultrasonic frequency to the resonant section of the stylet.

In some embodiments, the resonant section of the stylet is configured for decomposing the intraluminal clots by ultrasonic cavitation of fluid proximate of the intraluminal clots when the ultrasonic frequency is applied thereto by the ultrasound probe.

In some embodiments, the resonant section of the stylet is further configured for decomposing the intraluminal clots by ultrasonic agitation of a thrombolytic drug in the fluid proximate the intraluminal clots when the ultrasonic frequency is applied thereto by the ultrasound probe.

In some embodiments, the resonant section of the stylet is configured for decomposing the intraluminal clots by direct contact of the intraluminal clots with the stylet when the ultrasonic frequency is applied thereto by the ultrasound probe.

In some embodiments, the ultrasound probe is configured to automatically activate upon identification of the intraluminal clots by the changes in impedance in accordance with logic onboard a console to which the stylet and ultrasound probe are functionally connected in an operable state of the medical system.

In some embodiments, the ultrasound probe is further configured for ultrasound imaging of the intraluminal clots for characterization thereof.

In some embodiments, the ultrasound probe is further configured for ultrasound imaging of the intraluminal clots for confirming the patency in the catheter after the reestablishing thereof.

Also disclosed herein is a method of medical system for ultrasonic decomposition of intraluminal clots. The method includes a stylet-inserting step, a clot-identifying step, and a clot-decomposing step. The stylet-inserting step includes inserting the stylet into a lumen of a catheter. The clot-identifying step includes identifying an intraluminal clot in the catheter by detecting changes in impedance with one or more electrical impedance sensors in a distal portion of the stylet. The clot-decomposing step includes decomposing the intraluminal clot with ultrasound, thereby reestablishing patency in the catheter.

In some embodiments, the clot-decomposing step further includes decomposing the intraluminal clot by ultrasonic cavitation of fluid proximate of the intraluminal clot.

In some embodiments, the clot-decomposing step further includes decomposing the intraluminal clot by ultrasonic agitation of a thrombolytic drug in the fluid proximate the intraluminal clot.

In some embodiments, the method further includes a drug-injecting step. The drug-injecting step includes injecting the thrombolytic drug into the lumen of the catheter before ultrasonic agitation of the thrombolytic drug in the fluid proximate the intraluminal clot in the clot-decomposing step.

In some embodiments, the clot-decomposing step further includes decomposing the intraluminal clot by direct contact of the intraluminal clot with the stylet while one-or-more ultrasound transducers are in operation.

In some embodiments, decomposing the intraluminal clot with ultrasound includes using ultrasound provided by the one-or-more ultrasound transducers formed into an array of ultrasound transducers embedded in the distal portion of the stylet.

In some embodiments, decomposing the intraluminal clot with ultrasound includes applying an ultrasonic frequency to a resonant section in the distal portion of the stylet with an ultrasound probe.

These and other features of the concepts provided herein will become more apparent to those of skill in the art in view of the accompanying drawings and following description, which describe particular embodiments of such concepts in greater detail.

### DRAWINGS

FIG. 1 illustrates a medical system for ultrasonic decomposition of intraluminal clots in accordance with some embodiments.

FIG. 2 illustrates a distal portion of a stylet of the medical system including an ultrasound-transducer array in accordance with some embodiments.

FIG. 3 illustrates the distal portion of another stylet of the medical system including another ultrasound-transducer array in accordance with some embodiments.

FIG. 4 illustrates the distal portion of yet another stylet of the medical system including a resonant section of the stylet in accordance with some embodiments.

FIG. 5 illustrates decomposing an intraluminal clot to reestablish patency in a catheter with the ultrasound-transducer array of the stylet of FIG. 2 in accordance with some embodiments.

FIG. 6 illustrates decomposing an intraluminal clot to reestablish patency in a catheter with the resonant section of the stylet of FIG. 4 in accordance with some embodiments.

FIG. 7 illustrates a block diagram of the medical system in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, intraluminal clots can occur in lumens of catheters such as PIVCs, PICCs, CVCs, or the like, and such intraluminal clots are commonly treated with thrombolytic drugs such as alteplase using a 3-way stopcock method. While the 3-way stopcock method can be effective, it can also take over an hour or more to treat an intraluminal clot due to its size, extent of occlusion, and location in the catheter. Disclosed herein are medical systems and methods thereof for ultrasonic decomposition of intraluminal clots that improve upon at least the 3-way stopcock method.

### Medical Systems

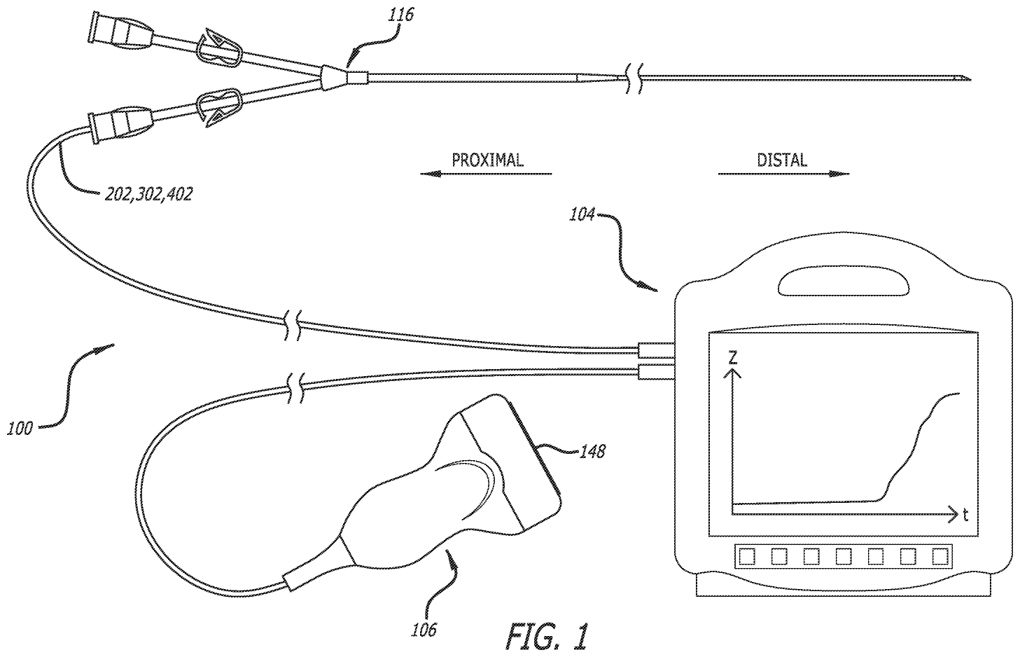

FIG. 1 illustrates a medical system 100 for ultrasonic decomposition of intraluminal clots in accordance with some embodiments.

As shown, the medical system 100 includes a stylet 202, 302, or 402 and, in some embodiments, a console 104 to which the stylet 202, 302, or 402 is connected in an operable state of the medical system 100. The medical system 100 can also include an ultrasound probe 106 functionally connected to the console 104 in an operable state of the medical system 100. Each of the foregoing components are described in more detail below beginning with the stylets 202, 302, and 402.

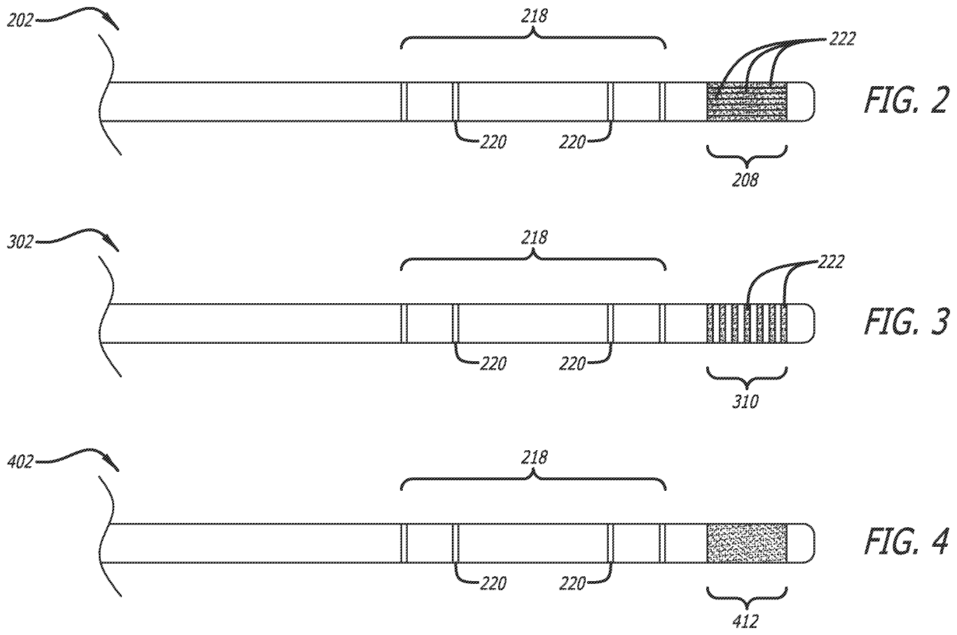

FIGS. 2-4 illustrate distal portions of the stylets 202, 302, and 402 of the medical system 100. FIG. 2 illustrates a distal portion of the stylet 202 of the medical system 100 including one or more ultrasound transducers 222 optionally in an ultrasound-transducer array 208 in accordance with some embodiments. FIG. 3 illustrates the distal portion of the stylet 302 of the medical system 100 including the one-or-more ultrasound transducers 222 optionally in another ultrasound-transducer array 310 in accordance with some embodiments. FIG. 4 illustrates the distal portion of the stylet 402 of the medical system 100 including a resonant section 412 of the stylet 402 in accordance with some embodiments.

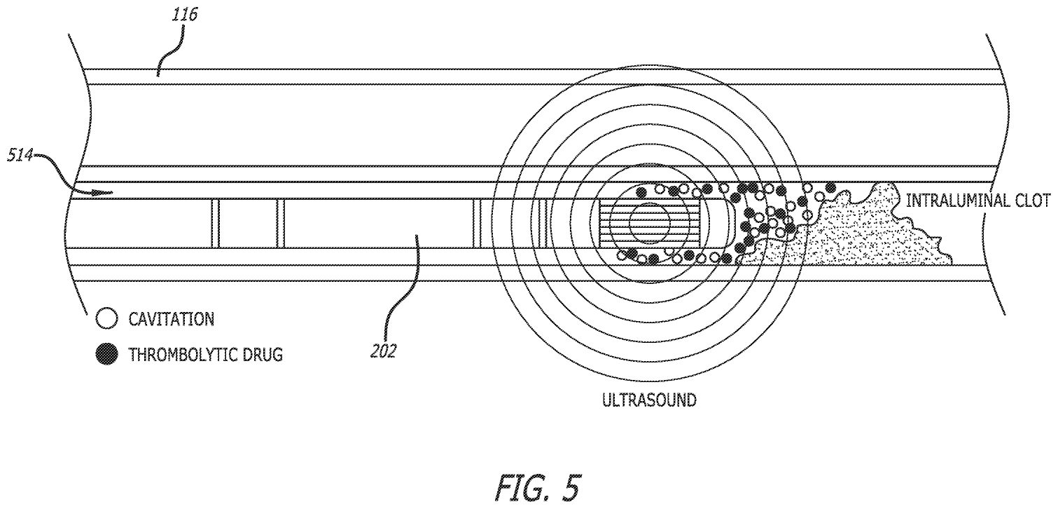

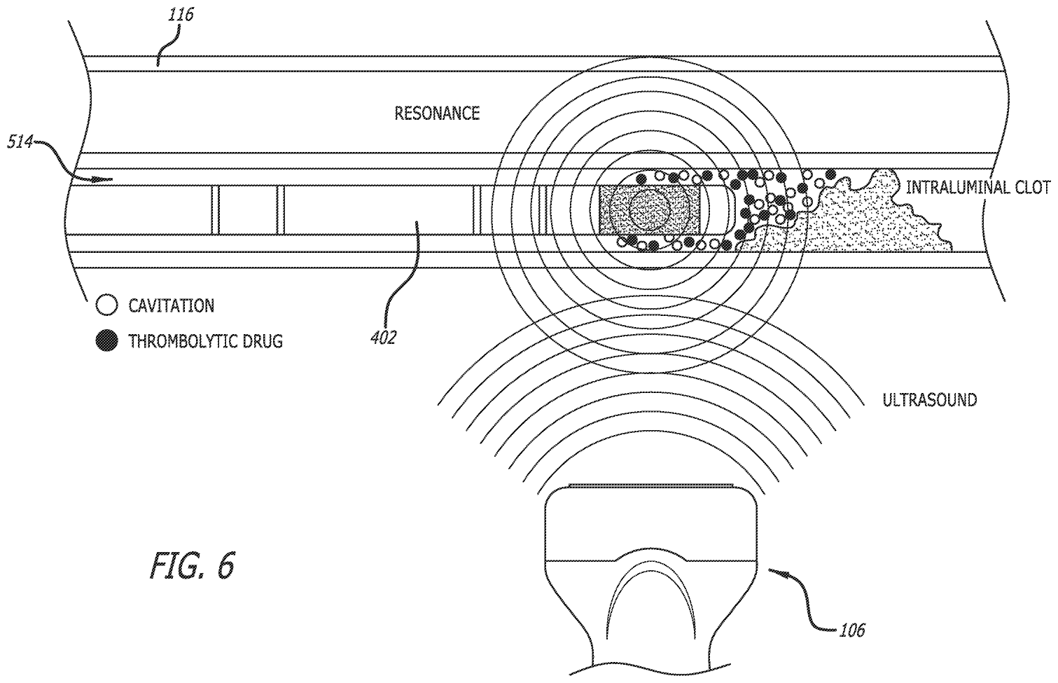

The stylet 202, 302, or 402 is configured to insert into a lumen 514 of a catheter 116 for identifying any intraluminal clots therein and, if present, ultrasonically decomposing the intraluminal clots. (See FIGS. 5 and 6.)

The stylet 202, 302, or 402 includes one or more electrical impedance sensors 220 such as ring electrodes optionally in an impedance-sensor array 218 in the distal portion of the stylet 202, 302, or 402. The one-or-more impedance sensors 220 are configured to detect changes in impedance for identifying any intraluminal clots in the catheter 116, and the one-or-more impedance sensors 220 can include any number of impedance sensors required for monopolar or multipolar (e.g., bipolar, tetrapolar, etc.) impedance measurements therefor.

The stylet 202 or 302 includes the one-or-more ultrasound transducers 222 optionally in the ultrasound-transducer array 208 or 310 embedded in the distal portion of the stylet 202 or 302 distal of the one-or-more impedance sensors 220 configured for decomposing any intraluminal clots in the catheter 116 and reestablishing patency thereof. FIG. 2 illustrates the one-or-more ultrasound transducers 222 formed into the ultrasound-transducer array 208 embedded along a length of the stylet 202, while FIG. 3 illustrates the one-or-more ultrasound transducers 222 formed into the ultrasound-transducer array 310 embedded around a circumference of the stylet 302. However, the one-or more ultrasound transducers 222 of either the stylet 202 or 302 can be in any arrangement required for decomposing any intraluminal clots in the catheter 116. Indeed, the one-or-more ultrasound transducers 222 can be in the ultrasound-transducer array 208 or 310 or another arrangement as required for decomposing any intraluminal clots in the catheter 116 by ultrasonic cavitation of fluid proximate the intraluminal clots, ultrasonic agitation of a thrombolytic drug (e.g., alteplase) in the fluid proximate the intraluminal clots, direct contact of the intraluminal clots with the stylet 202 or 302 while the one-or-more ultrasound transducers 222 are in operation, or a combination thereof.

As an alternative to the stylet 202 or 302 including the one-or-more ultrasound-transducers 222, the stylet 402 includes the resonant section 412 in the distal portion of the stylet 402 distal of the one-or-more impedance sensors 220 configured for decomposing any intraluminal clots in the catheter 116 and reestablishing patency thereof. The resonant section 412 of the stylet 402 is configured to resonate with an externally applied ultrasonic frequency for decomposing any intraluminal clots in the catheter 116. For example, the ultrasound probe 106 can be configured to apply the ultrasonic frequency to the resonant section 412 of the stylet 402. Indeed, the resonant section 412 of the stylet 402 can be configured for decomposing any intraluminal clots by ultrasonic cavitation of fluid proximate the intraluminal clots, ultrasonic agitation of a thrombolytic drug in the fluid proximate the intraluminal clots, direct contact of the intraluminal clots with the stylet 402, or the like when the ultrasonic frequency is applied to the resonant section 412 of the stylet 402 by the ultrasound probe 106.

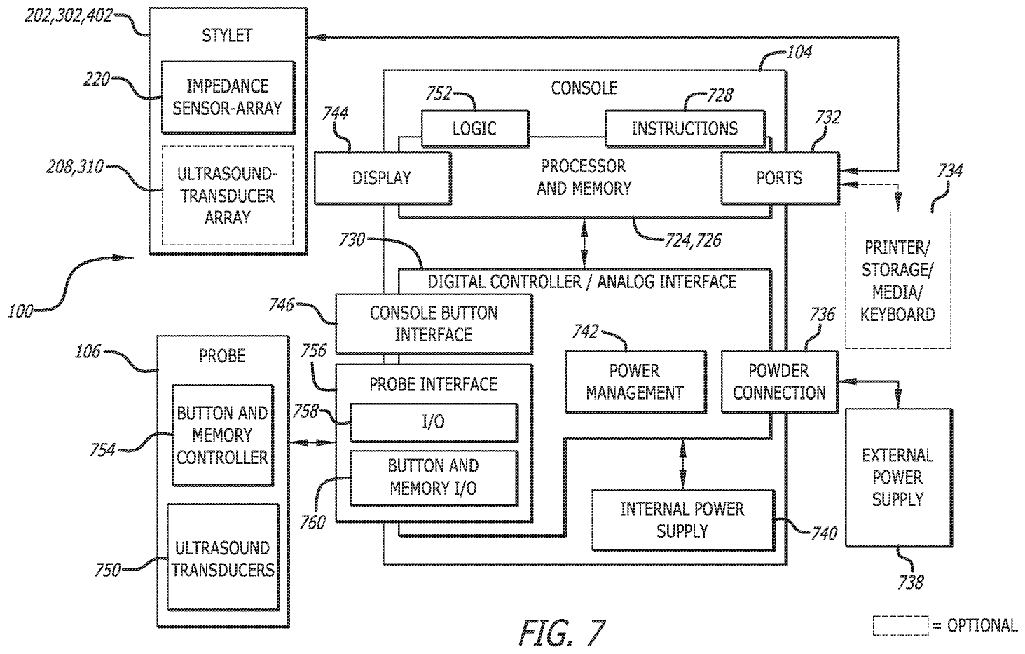

FIG. 7 illustrates a block diagram of the medical system in accordance with some embodiments.

As set forth above, the medical system 100 includes the stylet 202, 302, or 402, the optional ultrasound probe 106, and, in some embodiments, the console 104 to which the stylet 202, 302, or 402 and, when present, the ultrasound probe 106 are functionally connected in an operable state of the medical system 100. When the ultrasound probe 106 is present and functionally connected to the console 104, the medical system 100 is configured for ultrasound imaging of any intraluminal clots in the catheter 116 for characterization thereof. Advantageously, such a medical system can be used for confirming patency of the catheter 116 by ultrasound imaging after decomposing any intraluminal clots in the catheter 116 and reestablishing the patency thereof.

The console 104 houses and accommodates a variety of components of the medical system 100, and it is appreciated the console 104 can take any of a variety of forms. A processor 724 and memory 726 such as random-access memory (“RAM”) or non-volatile memory (e.g., electrically erasable programmable read-only memory [“EEPROM”]) is included in the console 104 for controlling functions of the medical system 100, as well as executing various logic operations or algorithms during operation of the medical system 100 in accordance executable instructions 728 therefor stored in the memory 726 for execution by the processor 724. For example, the console 104 is configured to instantiate by way of the instructions 728 one or more processes for identifying or decomposing any intraluminal clots in the catheter 116, as well as process electrical signals from the ultrasound probe 106 into ultrasound images for the ultrasound imaging. A digital controller/analog interface 730 is also included with the console 104 and is in communication with both the processor 724 and other system components to govern interfacing between the ultrasound probe 106 and other system components set forth herein.

The console 104 further includes ports 732 for connection with additional components such as the stylet 202, 302, or 402 and optional components 734 including a printer, storage media, keyboard, etc. The ports 732 can be universal serial bus (“USB”) ports, though other types of ports can be used for this connection or any other connections shown or described herein. A power connection 736 is included with the console 104 to enable operable connection to an external power supply 738. An internal power supply 740 (e.g., a battery) can also be employed either with or exclusive of the external power supply 738. Power management circuitry 742 is included with the digital controller/analog interface 730 of the console 104 to regulate power use and distribution.

A display screen 744 (e.g., a liquid-crystal display [“LCD”] screen) is integrated into the console 104 to provide a GUI and display information for a clinician during such as ultrasound images of intraluminal clots attained by the ultrasound probe 106. Alternatively, the display screen 744 is separate from the console 104 and communicatively coupled thereto. A console button interface 746 and control buttons included on the ultrasound probe 106 can be used to immediately call up a desired mode to the display screen 744 by the clinician for identifying or decomposing any intraluminal clots in the catheter 116.

The ultrasound probe 106 includes a probe head 148 that houses an array of ultrasound transducers 750, wherein the ultrasound transducers 750 are piezoelectric transducers or capacitive micromachined ultrasound transducers (“CMUTs”). The probe head 148 is configured for placement against skin of a patient over the catheter 116. In this way, the medical system 100, by way of the ultrasound probe 106 and logic 752, is able to characterize any intraluminal clots in the catheter 116 or confirm the patency of the catheter 116 by ultrasound imaging. Advantageously, the medical system 100 can be configured to automatically activate the one-or more ultrasound transducers 222 upon identification of any intraluminal clots by changes in impedance in accordance with the logic 752 onboard the console 104.

The ultrasound probe 106 also includes a button-and-memory controller 754 for governing button operation, as well as governing operation of the ultrasound probe 106. The button-and-memory controller 754 can include non-volatile memory (e.g., electrically erasable, programmable read-only memory [“EEPROM”]). The button-and-memory controller 754 is in operable communication with a probe interface 756 of the console 104, which includes an input/output (“I/O”) component 758 for interfacing with the ultrasound transducers 750 and a button-and-memory I/O component 760 for interfacing with the button-and-memory controller 754.

### Methods

FIG. 5 illustrates decomposing an intraluminal clot to reestablish patency in the catheter 116 with the one-or more ultrasound transducer 222 optionally in the ultrasound-transducer array 208 or 310 of the stylet 202 or 302 in accordance with some embodiments. FIG. 6 illustrates decomposing an intraluminal clot to reestablish patency in the catheter 116 with the resonant section 412 of the stylet 402 in accordance with some embodiments.

Methods of the medical systems set forth above include methods of using the medical systems. For example, a method of using the medical system 100 for ultrasonically decomposing intraluminal clots includes an optional stylet-connecting step, a stylet-inserting step, a clot-identifying step, and a clot-decomposing step.

The stylet-connecting step, when part of the method, includes functionally connecting the stylet 202, 302, or 402 to the console 104 as shown in FIG. 1.

The stylet-inserting step includes inserting the stylet 202, 302, or 402 into a lumen of a catheter such as the catheter 116 as shown in FIG. 5 for the stylet 202 and FIG. 6 for the stylet 402.

The clot-identifying step includes identifying an intraluminal clot in the catheter 116 by detecting changes in impedance with the one-or-more impedance sensors 220 in the distal portion of the stylet 202. 302, or 402.

The clot-decomposing step includes decomposing the intraluminal clot with ultrasound to reestablish patency in the catheter 116, which, in turn, can include decomposing the intraluminal clot by ultrasonic cavitation of fluid proximate the intraluminal clot, decomposing the intraluminal clot by ultrasonic agitation of a thrombolytic drug in the fluid proximate the intraluminal clot, decomposing the intraluminal clot by direct contact of the intraluminal clot with the stylet 202, 302, or 402 while the one-or-more ultrasound transducers 222 are in operation or the resonating section 412 of the stylet 402 is resonating with an ultrasonic frequency applied by the ultrasound probe 106, or a combination thereof.

The method can further include a drug-injecting step in association with the clot-decomposing step of decomposing the intraluminal clot by ultrasonically agitating the thrombolytic drug in the fluid proximate the intraluminal clot. The drug-injecting step includes injecting the thrombolytic drug into the lumen of the catheter 116 before ultrasonically agitating the thrombolytic drug in the fluid proximate the intraluminal clot in the clot-decomposing step.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical system for ultrasonic decomposition of intraluminal clots, comprising:
a stylet configured to insert into a lumen of a catheter including a fluid, the stylet including:
one or more electrical impedance sensors in a distal portion of the stylet, the one-or-more impedance sensors configured to detect changes in impedance for identifying intraluminal clots in the catheter; and
one or more ultrasound transducers embedded in the distal portion of the stylet, the one-or-more ultrasound transducers configured for decomposing the intraluminal clots to reestablish patency in the catheter.

2. The medical system of claim 1, wherein the one-or-more ultrasound transducers form an ultrasound-transducer array embedded along a length of stylet.

3. The medical system of claim 1, wherein the one-or-more ultrasound transducers form an ultrasound-transducer array are embedded around a circumference of the stylet.

4. The medical system of claim 1, wherein the one-or-more ultrasound transducers are configured for decomposing the intraluminal clots by ultrasonic cavitation of the fluid proximate of the intraluminal clots.

5. The medical system of claim 1, wherein the one-or-more ultrasound transducers are configured for decomposing the intraluminal clots by ultrasonic agitation of a thrombolytic drug in the fluid proximate the intraluminal clots.

6. The medical system of claim 1, wherein the one-or-more ultrasound transducers are configured for decomposing the intraluminal clots by direct contact of the intraluminal clots with the stylet while the one-or-more ultrasound transducers are in operation.

7. The medical system of claim 1, wherein the one-or-more ultrasound transducers are configured to automatically activate upon identification of the intraluminal clots by the changes in impedance in accordance with logic onboard a console to which the stylet is functionally connected in an operable state of the medical system.

8. A medical system for ultrasonic decomposition of intraluminal clots, comprising:
a stylet configured to insert into a lumen of a catheter including a fluid, the stylet including:
one or more electrical impedance sensors in a distal portion of the stylet, the one-or-more impedance sensors configured to detect changes in impedance for identifying intraluminal clots in the catheter; and
a resonant section of the stylet in the distal portion of the stylet distal of the one-or-more impedance sensors, the resonant section of the stylet configured to resonate with an applied ultrasonic frequency for decomposing the intraluminal clots to reestablish patency in the catheter; and
an ultrasound probe configured to apply the ultrasonic frequency to the resonant section of the stylet.

9. The medical system of claim 8, wherein the resonant section of the stylet is configured for decomposing the intraluminal clots by ultrasonic cavitation of the fluid proximate of the intraluminal clots when the ultrasonic frequency is applied thereto by the ultrasound probe.

10. The medical system of claim 8, wherein the resonant section of the stylet is configured for decomposing the intraluminal clots by ultrasonic agitation of a thrombolytic drug in the fluid proximate the intraluminal clots when the ultrasonic frequency is applied thereto by the ultrasound probe.

11. The medical system of claim 8, wherein the resonant section of the stylet is configured for decomposing the intraluminal clots by direct contact of the intraluminal clots with the stylet when the ultrasonic frequency is applied thereto by the ultrasound probe.

12. The medical system of claim 8, wherein the ultrasound probe is configured to automatically activate upon identification of the intraluminal clots by the changes in impedance in accordance with logic onboard a console to which the stylet is connected in an operable state of the medical system.

13. The medical system of claim 8, wherein the ultrasound probe is further configured for ultrasound imaging of the intraluminal clots for characterization thereof.

14. The medical system of claim 8, wherein the ultrasound probe is further configured for ultrasound imaging of the intraluminal clots for confirming the patency in the catheter after the reestablishing thereof.

15. A method of medical system for ultrasonic decomposition of intraluminal clots, comprising:
inserting the stylet into a lumen of a catheter including a fluid;
identifying an intraluminal clot in the catheter by detecting changes in impedance with one or more electrical impedance sensors in a distal portion of the stylet; and
decomposing the intraluminal clot with ultrasound, thereby reestablishing patency in the catheter.

16. The method of claim 15, wherein decomposing the intraluminal clot further includes decomposing the intraluminal clot by ultrasonic cavitation of the fluid proximate of the intraluminal clot.

17. The method of claim 15, wherein decomposing the intraluminal clot further includes decomposing the intraluminal clot by ultrasonic agitation of a thrombolytic drug in the fluid proximate the intraluminal clot.

18. The method of claim 17, further comprising injecting the thrombolytic drug into the lumen of the catheter before ultrasonic agitation of the thrombolytic drug in the fluid proximate the intraluminal clot.

19. The method of claim 15, wherein decomposing the intraluminal clot further includes decomposing the intraluminal clot with the stylet while one-or-more ultrasound transducers are in operation.

20. The method of claim 19, wherein decomposing the intraluminal clot with ultrasound includes using ultrasound provided by the one-or-more ultrasound transducers formed into an array of ultrasound transducers embedded in the distal portion of the stylet.

21. The method of claim 15, wherein decomposing the intraluminal clot with ultrasound includes applying an ultrasonic frequency to a resonant section in the distal portion of the stylet with an ultrasound probe.
